(19) United States
(12) Patent Application Publication
(10) Pub. No.: US 2014/0255354 A1
(43) Pub. Date: Sep. 11, 2014

(54) BARRIER CELL TREATMENT FOR MALIGNANT OR BENIGN TUMORS

(71) Applicant: Miguelangelo J. Perez-Cruet, Bloomfield, MI (US)

(72) Inventor: Miguelangelo J. Perez-Cruet, Bloomfield, MI (US)

(73) Assignee: MI4SPINE, LLC, Bloomfield Village, MI (US)

(21) Appl. No.: 13/793,635

(22) Filed: Mar. 11, 2013

(51) Int. Cl. A61K 35/32; C12N 5/071

(52) U.S. Cl. CPC A61K 35/32; C12N 5/0655
USPC 424/93.7; 604/522

(57) ABSTRACT

A method for administering a barrier cell treatment for malignant or benign tumor cells. The method includes preparing a material including chondrocytes and placing the material around the tumor cells. Preparing the material can include harvesting and culturing chondrocytes from the patient.

### BACKGROUND OF THE INVENTION

1. Field of the Invention

This invention relates generally to a method for administering chondrocytes to an area surrounding tumor cells and, more particularly, to a method for administering chondrocytes to an area surrounding tumor cells such that the chondrocytes act as a barrier that isolate and prevent nutrients and neovascularization, i.e., new tumor blood vessels, from reaching the tumor cells.

2. Discussion of the Related Art

According to recent data from the Center for Disease Control and Prevention, cancer remains the number two cause of death in America (23.27%), and is behind heart disease by a mere 0.87%, where approximately one in every four deaths each year is caused by cancer. Although there have been significant advances in the treatments for certain types of cancer, there has not been an effective treatment that minimizes damages to a patient caused by cancer and/or known cancer treatments.

Surgical oncology directly removes tumor cells from an organ through surgery. For example, a doctor can perform a lumpectomy to remove one or more portions of a breast for breast cancer, or perform a lobectomy to remove part of a lung for lung cancer. However, when treating non-resectable tumors for vital organs, such as brain, heart, pancreas, liver or kidney, surgery becomes virtually impossible because the surgery would likely result in significant harm or even possible death to the patient. As a result, when treating non-resectable malignant or benign tumors, doctors are often left with limited options, such as chemotherapy or radiation therapy, which are known to have significant side effects.

Tumor cells are known to vary in biological properties and characteristics, which leads to different causes, symptoms, and methods of treatment; hence the phrase, “there is no one single cure for cancer.” Despite these biological and characteristic differences, the different types of tumor cells tend to have one characteristic in common—that they proliferate through neovascularization, the process of recruiting new blood vessels for nutrients.

Neovascularization is a process of tissue vascularization that involves the growth of new blood vessels into a tissue. Tissue cells utilize blood vessels to supply oxygen and nutrients, and to remove waste products. Neovascularization of tumor cells plays a critical role in the development and progression of cancer. The process utilizes adjacent blood vessels to supply nutrients and oxygen, and correlates with cancer growth and its distant metastasis. Therefore, studies that involve cells and drugs that inhibit neovascularization of tumor cells have become the main focus for cancer treatments. Nevertheless, most known inhibitors still have side effects. Therefore, there is still a need in the art for finding an inhibitor that minimizes damage to the patient.

### SUMMARY OF THE INVENTION

In accordance with the teachings of the present invention, a method for administering a barrier cell treatment for malignant or benign tumor cells is disclosed. The method includes preparing a material including chondrocytes and placing the material around the tumor cells to surround the tumor cells. Preparing the material can include harvesting the chondrocytes from the patient or obtaining the chondrocytes from a same species donor (allograft) or a different species donor (xenograft).

Additional features of the present invention will become apparent from the following description and appended claims, taken in conjunction with the accompanying drawings.

### BRIEF DESCRIPTION OF THE DRAWINGS

FIG. 1 is a front view of a human patient including a brain, a pancreas and a liver;

FIG. 2 is a side view of an extraction needle inserted into a patient's spinal disc for extracting chondrocytes;

FIG. 3 is a perspective view of a culturing dish showing cultivation of the chondrocytes;

FIG. 4 is a perspective view of an injection needle inserted into a patient's liver;

FIG. 5 is a broken-away end view of a head portion of an injection needle; and

FIG. 6 is a broken-away cross-sectional end view of the head portion of the injection needle.

### DETAILED DESCRIPTION OF THE EMBODIMENTS

The following discussion of the embodiments of the invention directed to a method for a barrier cell treatment for malignant or benign tumors is merely exemplary in nature, and is in no way intended to limit the invention or its application or uses.

As discussed above, there is an interest in inhibiting neovascularization of tumor cells as an effective cancer treatment. As will be discussed in more detail below, one way to inhibit neovascularization is to surround the tumor cells with cells that do not contain blood vessels. In other words, these cells act as “siege cells” that isolate and inhibit tumor cells from obtaining nutrients and oxygen, which will then prevent the growth, progression and metastasis of tumor cells. Without the necessary nutrients to grow, the tumor cells are expected to die or at least not multiply as a result of the isolation.

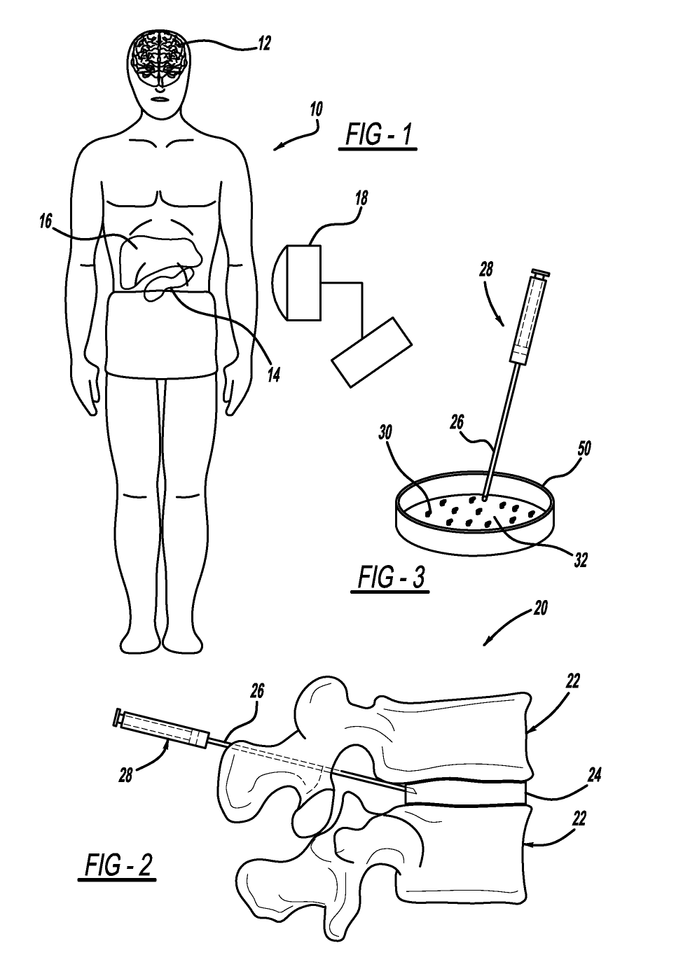

FIG. 1 is a perspective view of a human patient 10 having a brain 12, a pancreas 14 and a liver 16. The brain 12, the pancreas 14 and the liver 16 are typically the organs or locations in the human body where surgically removing tumor cells is deemed difficult or too risky in terms of the patient's health. A person skilled in the pertinent art is able to locate tumor cells using imaging studies such as MR imaging (with and without contrast), spectroscopy imaging, PET scanning, or CT scanning (with and without contrast), using, for example, an imager 18. These imaging technologies are effective in locating tumors and their boundaries.

Once tumors and their boundaries have been located, chondrocytes are then extracted from an individual patient's body (autograft) or a tissue culture from a same species donor (allograft) or a different species donor (xenograft), as discussed in detail below. Chondrocytes or cartilage cells are found in the middle of spinal intervertebral discs, and make up the nucleus pulposus. Chondrocytes offer several unique advantages as barrier cells. For example, chondrocytes do not require vascular support, meaning that chondrocytes maintain their viability and survivability in a different manner from other cells because chondrocytes obtain nutrients and oxygen and remove their waste products through “diffusion.” Chondrocytes obtain all of their nourishment from blood vessels in the adjacent vertebral end plate perichondrium and surrounding connective tissues by diffusion of oxygen and nutrients without direct blood supply to the chondrocytes. They thrive in a low oxygen environment. Furthermore, chondrocytes potentially possess an immune privileged property, which reduces immune rejection when administered into the patient's viable organs. Therefore, these unique biological characteristics can enable the chondrocytes, if placed around a tumor, to effectively isolate and inhibit tumor cells from progression and metastasis.

FIG. 2 is a side view of a portion of a human spine 20 including two vertebrae 22 separated by a spinal disc 24. An extraction syringe 28 having a needle 26 is inserted into the disc 24 for extracting chondrocytes therefrom.

Once the desired amount of chondrocytes are extracted or harvested, the chondrocytes are then cultured according to procedures known in the art. FIG. 3 is a perspective view of a culturing dish 28 receiving the chondrocytes 30 from the syringe 28 that have been harvested from the disc 24. The chondrocytes 30 are cultured in a culturing medium 32 where they are grown for tumor treatment in a manner well understood by those skilled in the art.

As mentioned, the source of the chondrocytes is not limited to the patient 10. Although it is more preferential to use the patient's own chondrocytes for eliminating the risk of an immune response, allografts from other human donors may also be used to extract chondrocytes to treat the patient 10. Furthermore, if the target tumor cells are located in an immune-privileged site, such as the brain 12, where brain cells are able to tolerate foreign cells without inflammatory immune response, then potentially xenograft chondrocytes from animals (such as bovine or porcine) can be used.

In addition, stem cell technology can be utilized to provide the necessary chondrocytes for this treatment. Human embryonic stem cells can be taken from bone marrow, fat tissue, skin, umbilical cord blood, or any other sources of embryonic stem cell, and can then be harvested, treated, and differentiated according to known procedures in the art into chondrocytes for administering the treatment.

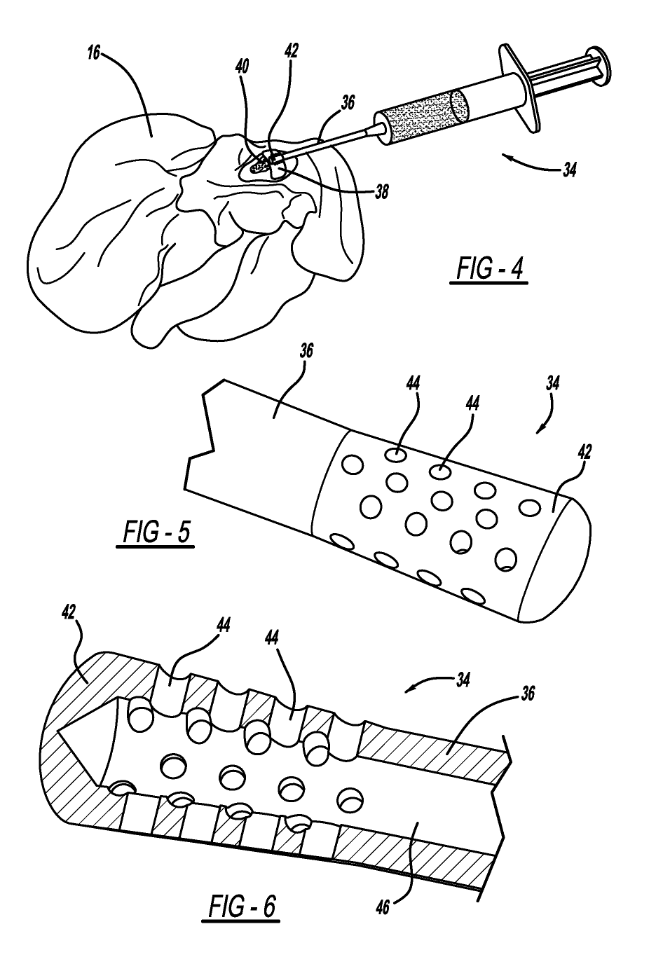

After the chondrocytes are harvested and cultured, they are administered into the patient's organ where tumor cells are located. FIG. 4 is a perspective view of a syringe 34 including an injection needle 36 for administering cultured chondrocytes 38 in a manner that encircles or surrounds tumor cells 40 inside the patient's liver 16. The needle 36 includes a head portion 42 for selectively administering a layer or layers of the cultured chondrocytes 38 over the tumor cells 40. Alternately, multiple injection pathways can be used to inject the cultured chondrocytes 38 around the tumor cells 40.

FIG. 5 is a broken-away view and FIG. 6 is a broken-away cross-sectional view of the injection needle 36 showing the head portion 42. The head portion 42 includes multiple openings 44 for effectively administering the cultured chondrocytes 38. The openings 44 are in fluid communication with a center channel 46 of the injection needle 36 and the injection syringe 34. When the syringe 34 pushes the cultured chondrocytes 38 into the channel 46, the cultured chondrocytes 38 will exit through the openings 44 to fully surround the tumor cells 40. Those skilled in the art will be able to determine the number, size, orientation, etc. of the openings 44 and the size of the needle 36 for a particular tumor or a particular organ. Further, those skilled in the art will readily understand that other types of diffusion catheters can be used.

Depending on how the cultured chondrocytes 38 react with the soft tissues surrounding the tumor cells 40, it is possible to achieve isolation in one administration. For example, if the tumor cells 40 cease to recruit the surrounding blood vessels, isolation is then completed. After administering the cultured chondrocytes 38 to the tumor cells 40, isolation of the tumor cells 40 is verified. For example, in order to visualize the isolation of the cells 40, the cells may be labeled with radioactive substance, dye, contrast, or a series of tracers, such that it may be determined that the tumor cells 40 have been isolated. However, if the tumor cells 40 continue to recruit the surrounding blood vessels, multiple injections of the cultured chondrocytes 38 may be needed to fully isolate the tumor cells 40. Alternately, if imaging determines that the tumor cells 40 are not isolated at a particular point, additional treatment can be performed.

A plurality of layers of the cultured chondrocytes 38 may be administered, where the thickness of the chondrocyte layers vary from millimeters to possible centimeters depending on the number and size of the tumor cells 40. The necessary depth of the cultured chondrocytes 38 will be at least enough to encircle and isolate the entire group of the tumor cells 40 and potentially kill the tumor cells 40 or keep them from growing.

In an alternative embodiment, a specialized cannula (not shown) may be used to allow directional injections for circumventing the tumor 360 degrees in order to encircle the tumor cells 40. In another alternative embodiment, the cultured chondrocytes 38 are administered to four quadrants of the tumor cells 40 for isolation. In other words, the cultured chondrocytes 38 are administered to the upper-left, upper-right, lower-left, and lower right quadrant of the tumor cells 40. In addition, depending on the organ where the tumor cells 40 are located, an array of deliver devices may be utilized to achieve full containment of the tumor cells 40. For example, specialized needles for brain insertion may be used to treat brain tumors, or specialized needles for pancreas insertion may be used to treat pancreatic tumor.

The foregoing discussion discloses and describes merely exemplary embodiments of the present invention. One skilled in the art will readily recognize from such discussion and from the accompanying drawings and claims that various changes, modifications, and variations can be made therein without departing from the spirit and scope of the invention as defined in the following claims.

What is claimed is:

1. A method for administering a barrier cell treatment for malignant tumors, said method comprising:
identifying a location of tumor cells in a body part;
preparing a tumor treatment material that includes chondrocytes; and
administering the tumor treatment material around the tumor cells to isolate the tumor cells.

2. The method according to claim 1 further comprising labeling the tumor treatment material with a radioactive substance, dye, imaging contrast agent or a plurality of cell markers before the material is administered.

3. The method according to claim 2 further comprising verifying the isolation of the tumor cells using the labeling.

4. The method according to claim 1 wherein preparing the tumor treatment material includes extracting chondrocytes from a human spine.

5. The method according to claim 1 wherein preparing the tumor treatment material includes extracting chondrocytes from a non-human spine.

6. The method according to claim 1 wherein preparing the tumor treatment material including extracting the chondrocytes from tissue culture

7. The method according to claim 1 wherein preparing the tumor treatment material including from stem cell differentiated chondrocytes.

8. The method according to claim 5 wherein preparing the tumor treatment material includes extracting chondrocytes from an animal spine.

9. The method according to claim 5 wherein preparing the tumor treatment material further includes harvesting the chondrocytes that are extracted from the non-human spine.

10. The method according to claim 1 wherein administering the tumor treatment material includes administering the tumor treatment material by an injection.

11. The method according to claim 8 wherein administering the tumor treatment material includes administering the tumor treatment material via an injection needle that includes a plurality of openings in a needle head portion.

12. The method according to claim 1 wherein administering the tumor treatment material including the chondrocytes includes utilizing a specialized cannula or catheter to circumvent the tumor cells to maximize isolation of the tumor.

13. The method according to claim 1 wherein administering the tumor treatment material includes administering the tumor treatment material to four quadrants of the tumor cells to isolate the tumor cell.

14. The method according to claim 1 wherein preparing the tumor treatment material includes differentiating and harvesting embryonic stem cells into chondrocytes.

15. The method according to claim 12 wherein administering the tumor treatment material includes administering the differentiated embryonic stem cells to the upper-left, upper-right, lower-left, and lower-right quadrants of the tumor cells.

16. The method according to claim 1 wherein the body part is a brain, pancreas or liver or a plurality of vital organ systems.

17. A method for administering a barrier cell treatment for malignant tumors in a human, said method comprising:
identifying a location of tumor cells in a body part of the human;
harvesting chondrocytes from a spinal disc of the human;
culturing the harvested chondrocytes in a culturing medium to grow the chondrocytes; and
administering the cultured chondrocytes to the body part so that the cultured chondrocytes at least partially surround the tumor cells to isolate the tumor cells.

18. The method according to claim 15 further comprising labeling the cultured chondrocytes with a radioactive substance, dye, imaging contrast agent or a plurality of cell markers before the chondrocytes are administered.

19. The method according to claim 15 wherein administering the cultured chondrocytes includes administering the cultured chondrocytes by an injection.

20. The method according to claim 17 wherein administering the cultured chondrocytes includes administering the cultured chondrocytes via an injection needle that includes a plurality of openings in a needle head portion.

21. The method according to claim 15 wherein the body part is a brain, pancreas or liver and or other vital organ system.

22. A method for administering a barrier cell treatment for malignant or benign tumors in a human, said method comprising:
identifying a location of tumor cells in a body part of the human;
harvesting chondrocytes from a donor;
culturing the harvested chondrocytes in a culturing medium to grow the chondrocytes; and
injecting the cultured chondrocytes into the body part using an injection needle that includes a plurality of openings in a needle head portion so that the cultured chondrocytes at least partially surround the tumor cells to isolate the tumor cells.
